(19) United States
(12) Patent Application Publication
(10) Pub. No.: US 2005/0172970 A1
(43) Pub. Date: Aug. 11, 2005

(54) Hemi-cannula for tracheotomy patients

(76) Inventor: Luis Blas Mompo Orti, Valencia (ES)

Correspondence Address: RICHARD M. GOLDBERG, 25 EAST SALEM SREEET, SUITE 419, HACKENSACK, NJ, 07601, US

(21) Appl. No.: 10/514,396

(22) PCT Filed: May 13, 2002
(86) PCT No.: PCT/ES02/00225

(51) Int. Cl. A62B 9/06; A61M 16/00

(52) U.S. Cl. 128/207.14; 128/207.15

(57) ABSTRACT

A hemi-cannula for tracheotomy patients, includes a main tubular body (1.1) which is made from a suitable flexible material and which includes peripheral end fixing flanges (1.2 and 1.3). One of the flanges (1.2), which is intended to be disposed inside the trachea next to the inner face of same, takes the form of a wing including a cylindrical surface with an oblique axis in relation to an axis perpendicular to the main axis of the main body (1.1). The other flange (1.3) takes the shape of a truncated cone including a larger outer base. A conduit forming the main tubular body passes through the center of both of the flanges.

### OBJECT OF THE INVENTION

The object of this invention is a hemi-cannula for tracheotomy patients of the sort used for reinforcing the trachea, allowing the opening to be closed, preventing air loss and facilitating phonation.

Throughout this explanation we will use the term cannula to refer both to cannulas with an internal extension and to hemi-cannulas in which said internal extension is not present.

One of its characteristic features is a flexible perimeter flange adapted to the inner side of the larynx to prevent this from coming out.

It is similarly characterised by the fact that the outer end has a conical shape to prevent the cannula from going into the opening to ensure hermetic sealing of the tracheostoma.

Lastly, it also has the characteristic of a perimeter projecting rim on the outer edge of the lumen to facilitate closing during phonation.

### BACKGROUND TO THE INVENTION

A tracheotomy consists in opening the trachea directly, with this coming directly out through the skin in the front of the neck.

A tracheotomy is performed through a problem of obstruction in the larynx which prevents the patient from getting sufficient ventilation, so a secondary channel for air inlet to the trachea is made without going through the glottis.

The tracheotomy patient needs to have a cannula fitted to reinforce the opening made connecting the trachea with the exterior to prevent this from closing and thus blocking or reducing the passage of air during breathing.

The presence of a tracheotomy does not necessarily mean that the sick person is unable to speak, as in many cases phonation is possible by blocking the tracheostoma, so that the air forced out goes through the larynx.

This means that there are cases in which the air breathed out going through the larynx is sufficient to talk even though insufficient for inhaling.

The problem of these patients fitted with a traditional type cannula, required for ventilation, is that when this is closed the air breathed out to phonate must go through the space left between the cannula, a foreign body in the trachea and the walls of the trachea itself.

Furthermore, part of the air circulating escapes through the tracheostoma which is not hermetically sealed.

To mitigate this problem fenestrated cannulas have been designed that only allow through the air exhaled through the cannula towards the larynx.

One of the cannulas commonly used is a silver cannula which is inserted in the larynx and which closes the tracheostoma by means of a cloth.

This type of cannula produces great suffering for the patient since, when the back is turned, the cannula scrapes against the internal wall of the trachea, even producing lesions.

EP 0691136 discloses a cannula which makes use of this type of configuration and includes a ring element as a reinforcement device as well as a speaking valve.

There is another type of silicon valves which are not so invasive as they have an internal flange which fits the internal side of the trachea but they nevertheless require a cover to be fitted so that the patient can talk when air is expelled.

In this cannula, it is necessary to remove the cover during expectoration to clean this and then put it back in.

ES 2005267 describes a cannula with a securing ball which is housed inside the trachea and which requires a collar or similar device outside to hold it to the neck.

Ball cannulas ensure the seal between the trachea, and the cannula and are used above all in deep probes.

The ideal solution consists of a non-irritating element which only acts as a retainer of the tracheostoma with no need to insert a foreign body in the trachea.

It should also keep the tracheal opening unchanged to allow air freely through both when breathing in and out so as not to prevent phonation by means of hermetic blocking of the tracheostoma.

It is also useful for the device not to require the use of tapes to secure this to the neck.

Both criteria are fully complied with in the present invention.

### DESCRIPTION OF THE INVENTION

The invention covered by this description refers to a hemi-cannula for tracheotomy patients which allows the air through, reinforcing the tracheostoma at the same time as not preventing phonation.

This cannula consists of a single body made of a tolerable and flexible material, such as silicon, formed mainly of a central tube.

The shape of the tube section will depend on the shape of the tracheostoma, though it is desirable for it to take a circular or elliptical shape.

This cylindrical body allows the tracheostoma to be kept open without breaking the passage of air through.

To secure the cannula, any possible movement both towards the outside and the inside has to be prevented.

To prevent the hemi-cannula from coming out of its location, for example through expectoration, it has a membrane on the inside end like a flange, which sticks out transversely and obliquely from the body forming the main tube of the hemi-cannula.

Through following the generatrix of a cylindrical surface, it adapts to the internal walls of the trachea, leaving the whole tracheal opening free both when breathing in and out.

The fact of the axis of the directrix of the cylindrical surface being oblique allows the angle of the trachea to adapt in respect of the main axis of the tracheostoma.

The opposite end of the hemi-cannula, that is, the end which is placed outside, has an essentially truncated cone shape, with a base greater towards the outside, which is crossed by the conduit forming the hemi-cannula. The greater section will prevent accidental insertion into the trachea, facilitating proper closing of the tracheostoma at the same time.

The conical wall preferentially has certain convexity. The convexity of this projecting shoulder keeps the conicity and favours closing even when it is compressed through the effect of the pressure exerted by the internal side of the tracheostoma.

The base of the cone is centred in respect of the main axis of the internal opening and has a small projection on its inner edge which facilitates hermetical closure when it is blocked with a finger when talking.

Given that the two projecting shoulders prevent the cannula from moving, it is not necessary to use tapes or other items for securing to the patient's neck.

The arrangement in a circular or elliptical section of the main tube, the distances between projecting shoulders and the angles of the conical projecting shoulder and the generating line of the internal flanges vary depending on the size of the tracheostoma and its geometry.

### DESCRIPTION OF THE DRAWINGS

This descriptive report is complemented with a set of drawings illustrating the preferential example and not limiting the invention at all.

FIG. 1 is a perspective in which the elevation, profile and plan view of the cannula are shown.

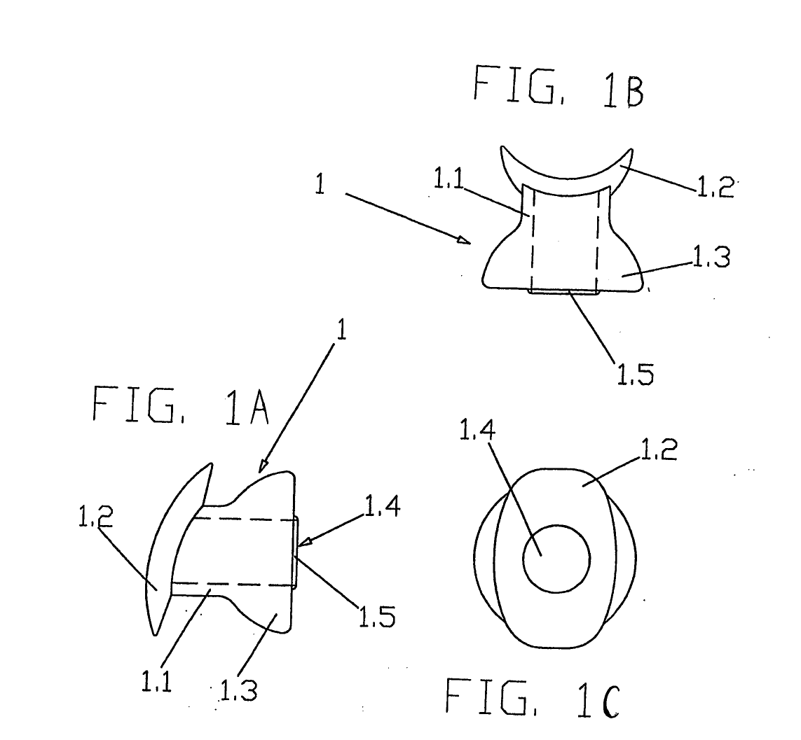

FIG. 2 is a representation of the cannula shown in the profile of a patient.

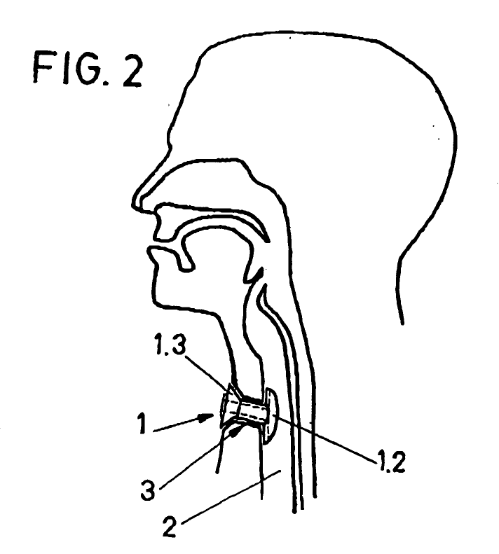

### PREFERENTIAL EMBODIMENT OF THE INVENTION

In view of what is stated above, this invention refers to a cannula for tracheotomy patients.

This cannula (1) has a main tubular body (1.1) with a preferentially circular or elliptical section with two projecting shoulders (1.2) and (1.3) at the ends

One first projecting shoulder (1.2) which corresponds to the inside of the trachea (2) which has a flange shape with a cylindrical surface, whose directrix cylinder is oblique in respect of the direction perpendicular to the axis of the main body (1.1).

The internal projecting shoulder (1.2) adapts to the internal wall of the trachea (2) leaving its lumen open.

The opposite end, coinciding with the front part of the neck, has a conical perimeter curved-convex shoulder (1.3) which prevents the cannula (1) from getting inside the patient through the tracheostoma (3).

The slight convexity of this projecting shoulder (1.3) absorbs any deformation which the material might undergo doe to the pressure required to keep the cannula secured (1), the resulting deformation of this surface being close to a conical shape.

The conduit (1.4) communicates the trachea (2) with the outside.

In the event of wishing to close the conduit (1.4) in order to speak, the cannula (1) has a perimeter projecting rim (1.5) on the internal side which facilitate closing without leaks with slight finger pressure.

The flexibility of the material facilitates the extraction for cleaning both the cannula (2) and the tracheostoma (3).

The circular edge which forms the outer base of the trunk of the cone is rounded off to prevent it from being aggressive to the patient, as well as having edges that are also preferentially rounded off in the changes of section of the different parts of the cannula.

The essential nature of this invention is not affected by any variations in material, shape, size and arrangement of its component items, described in a non-limiting way, this being enough to proceed to its reproduction by an expert.

For application in manufacturing cannulas for tracheotomy.

1. Hemi-cannula for tracheotomy patients, comprising:
a main tubular body of flexible material, said tubular body having a main axis;
two end perimeter projecting shoulders at opposite ends of said tubular body for securing the hemi-cannula to a trachea;
an internal one of said shoulders adapted to be placed inside the trachea, adjacent to an internal side of the trachea, said one shoulder having a flange shape with a cylindrical surface and with an axis which is oblique in respect to a plane perpendicular to the main axis of the body;
an outer one of said shoulders having a shape of a truncated cone, with a larger outer base; and
an air passage conduit which extends through the main tubular body and both shoulders.

2. Hemi-cannula for tracheotomy patients, according to claim 1, wherein the truncated conical outer shoulder has a curved convex shape lateral surface.

3. Hemi-cannula for tracheotomy patients, according to claim 1, wherein the large outer base of the truncated conical outer shoulder forms an outer projecting shoulder which has a projecting rim on an inner edge thereof through which the air passage conduit extends.

4. Hemi-cannula for tracheotomy patients, according to claim 3, wherein the projecting rim on the inner edge at the outer shoulder has a sharp edged outer section.

5. Hemi-cannula for tracheotomy patients, according to claim 1, wherein the internal shoulder has a blunt perimeter edge.

6. Hemi-cannula for tracheotomy patients, according to claim 1, wherein the main tubular body has a cylindrical cross-section.

7. Hemi-cannula for tracheotomy patients, according to claim 1, wherein the main tubular body has an elliptical cross-section.

8. Hemi-cannula for tracheotomy patients, according to claim 1, wherein the main tubular body has an oval cross-section.
